(19) United States
(12) Patent Application Publication
(10) Pub. No.: US 2002/0032197 A1
(43) Pub. Date: Mar. 14, 2002

(54) Methods and compositions for using moclobemide

(76) Inventors: Donald F. Klein, New York, NY (US); Seth Lederman, New York, NY (US)

Correspondence Address: FISH & NEAVE, 1251 AVENUE OF THE AMERICAS, 50TH FLOOR, NEW YORK, NY, 10020-1105, US

(21) Appl. No.: 09/772,679

(22) Filed: Jan. 30, 2001

Related U.S. Application Data
(63) Continuation of application No. PCT/US99/17274, filed on Jul. 31, 1999.
(60) Provisional application No. 60/094,934, filed on Jul. 31, 1998.
(60) Provisional application No. 60/094,984, filed on Jul. 31, 1998.
(60) Provisional application No. 60/094,985, filed on Jul. 31, 1998.
(60) Provisional application No. 60/094,987, filed on Jul. 31, 1998.
(60) Provisional application No. 60/094,989, filed on Jul. 31, 1998.

(51) Int. Cl. A61K 31/535

(52) U.S. Cl. 514/237.8

(57) ABSTRACT

The invention relates to methods and compositions for treating, managing, and/or preventing certain pain and pain disorder, posttraumatic stress disorder (PTSD), premenstrual dysphoric disorder and premenstrual syndrome, certain sleep disorders, eating disorders, and symptoms thereof using moclobemide, a moclobemide metabolite, a moclobemide derivative or a moclobemide composition.

This application incorporates by reference herein U.S. application No. 60/094,985; U.S. application No. 60/094,987; U.S. application No. 60/094,984; U.S. application No. 60/094,934; and U.S. application No. 60/094,989; all of which were filed on Jul. 31, 1998.

### FIELD OF THE INVENTION

The invention relates to methods of treatment, management, and/or prevention of certain pain and pain disorder, posttraumatic stress disorder (PTSD), premenstrual dysphoric disorder and premenstrual syndrome, certain sleep disorders, eating disorders, and symptoms thereof.

### BACKGROUND OF THE INVENTION

### Chemistry and Pharmacokinetics of Moclobemide

Moclobemide, or p-chloro-N-(2-morpholinoethyl)-benzamide, which is represented by the formula:

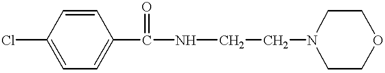

is described in U.S. Pat. No. 4,210,754, to Burkard et al.

Moclobemide is a selective and reversible monoamine oxidase (MAO) subtype A inhibitor. Kettler, R. et al., 1990, Acta Psychiatr. Scand., Supp. 360(82):101-103. Unlike other monoamine oxidase inhibitors, which irreversibly and non-selectively bind MAO and can have severe food and drug interactions that limit their therapeutic utility, moclobemide is part of a distinct class of selective MAO inhibitors that inhibit predominantly or selectively either monoamine oxidase A (MAO-A) or monoamine oxidase B (MAO-B). Compounds that selectively inhibit MAO-B, and thereby inhibit the degradation of dopamine, are useful for the treatment of neurological and neurodegenerative diseases of the dopaminergic pathway, such as Parkinson's disease. Livingston, M. G. and Livingston, H. M., 1996, Drug Safety, 14(4):218-227. Compounds that selectively inhibit MAO-A predominantly affect the degradation of serotonin and norepinephrine, leading to increased concentrations of these neurotransmitters in synapses, and are useful for depression. Livingston and Livingston, 1996.

The in vitro binding of moclobemide to MAO-A is weak, but more than 167-fold more selective than for the MAO-B isozyme. The ex vivo binding of moclobemide to MAO-A has been demonstrated to be reversible, with sufficient dissociation to result in recovery of enzyme activity within 16 hours. Fulton, B. and Benfield, P., 1996, Drugs, 52(3):451. This is in contrast to older, nonselective and irreversible MAO inhibitors (also referred to herein as “irreversible MAOIS” or “IMAOIs”), which irreversibly bind to either or both MAO-A and MAO-B isozymes and exhibit enzyme inhibition lasting several days. Da Prada, M. et al., 1990. As the MAO-A inhibition in vivo is relatively short in duration, it is generally accepted to be reversible. Da Prada et al., 1990, Acta Psychiatr. Scand., Suppl. 360(82):103-105.

The effects of moclobemide on monoamine metabolism and/or activity of monoaminergic neurons have been indirectly demonstrated in humans by reductions in plasma levels of the catecholamine metabolites homovanillic acid, 3,4-dihydroxyphenylacetic acid, and 3-methoxy-4-hydroxyphenylglycol and the serotonin (5-hydroxytryptamine) metabolite 5-hydroxyindoleacetic acid. In vitro, moclobemide has no appreciable affinity for muscarinic, dopaminergic, serotonergic, adrenergic, H₁-histaminergic, benzodiazepine or opioid receptors. Da Prada et al., 1981, Excerpta Medica, 183-196; Da Prada et al., 1983, Mod. Probl. Pharmacopsych., 19:231-245; Da Prada et al., 1984, Clin. Neuropharmacol., 7 (Suppl. 1):684-685.

Moclobemide is extensively distributed in the body and rapidly eliminated from plasma by metabolic conversion in the liver. After single-dose oral administration, moclobemide is almost completely absorbed; however, bioavailability ranges from 44% to 69% because of substantial first-pass metabolism. Guentert, T. W. et al., 1990, Acta Psychiatr. Scand., Suppl. 360(82):91-93. After multiple administrations, moclobemide displays increased bioavailability (approx. 85%), possibly due to saturation of first-pass metabolism. Id. Moclobemide is metabolized to at least 19 different metabolites, one of which has moderate MAO-A inhibitory activity. Jauch, R. et al., 1990, Acta Psychiatr. Scand., Suppl. 360(82):87-90. Age and renal function are reported to have no significant effect on the pharmacokinetics of moclobemide; however, elimination is impaired in patients with hepatic dysfunction. Stoeckel et al., 1990, Acta Psychiatr. Scand., Suppl. 360(82):94-97. Thus, patients suffering from liver impairment should receive only 50% of the normal dosage. Id.

One of the most attractive features of moclobemide is its impressive safety profile. Because it does not permanently inhibit the monoamine oxidase enzyme, it has a relatively brief pharmacological action that contributes to its clinical safety. Moclobemide's short plasma half life (1 to 2 hours) also contributes to its safety because it promptly degrades in the tissues thereby preventing local over-accumulations. Stoeckel et al., 1990. Moclobemide appears to produce fewer adverse events in normal clinical use than other reversible MAOI compounds. Priest, R.G., et al., 1995, J. Clin. Psychopharm., 15(4), Suppl. 2: 1S-3S. The most frequently reported adverse events were psychiatric, neurologic, and gastrointestinal disorders, with hepatobiliary events occurring only rarely (Hilton, S. et al., 1995, J. Clin. Psychopharmacol., 15(4 Suppl. 2):76S-83S), and the effects of moclobemide on the sleep of healthy volunteers appear weak in comparison to other MAO inhibitors. Blois, R. et al., 1990, Acta Psychiatr. Scand., Suppl. 360(82):73-75.

Although moclobemide has not been extensively used in the United States, it has been used in Europe and in other countries and it is not known to produce any clinically relevant interactions with commonly prescribed drugs. Zimmer, R. et al., 1990, Acta Psvchiatr. Scand., Suppl. 360(82):84-86. Moreover, moclobemide is far less likely than traditional MAO inhibitors to induce hypertensive reactions with the concomitant administration of sympathomimetic drugs, or consumption of tyramine-rich foods. Hilton, S. E., 1997, Eur. Arch. Psychiatry Clin. Neurosci., 247:113-119; Zimmer, 1990, Acta Psychiatr. Scand., Suppl. 360(82):81-83; Zimmer, Fischbach et al., 1990, Acta Psychiatr. Scand., Suppl. 360(82):76-77; Zimmer, Puech et al., 1990, Acta Psychiatr. Scand., Suppl. 360(82):78-80; Da Prada et al., 1990, Acta Psychiatr. Scand., Suppl. 360(82):106-107. A number of studies also suggest that moclobemide is better tolerated than other compounds with anti-depressant activity. Priest, R. G., et al., 1995. Perhaps most impressively, the fatal toxicity index of moclobemide approaches zero. Hilton et al., 1995.

Initially, the recommended doses for moclobemide therapy (i.e., to treat depression) were approximately 100 mg to 150 mg three times daily. Guentert, T. W. et al., 1990. Subsequent experience has suggested that, in view of the positive dose-response curve found with moclobemide, doses as high as 600 mg daily are increasingly efficacious and remain well-tolerated. Fitton, A. et al., 1992, Drugs, 43(4):561-596.

Moclobemide's excellent safety and positive tolerance profile have made it a popular anti-depressive in Canada, Europe, Australia, New Zealand, South Africa, and Latin America. Angst J. et al., 1996, Int. Clin. Psychopharmacol., 11(Suppl. 3):3-7; Glick, I. D. et al., 1995 Schatzberg, A. F. and Nemeroff, C. B. (Ed.), The American Psychiatric Press Textbook of Psychopharmacology, Washington, D.C., pp. 839-846)). Prescribing medications such as irreversible MAOI's with potentially harmful or deadly side effects to treat depression has traditionally caused some measure of concern among physicians, who worry that depressed patients may attempt suicide with the very medicines they are prescribing. Although moclobemide is an MAO inhibitor, as an anti-depressive, it appears to be less prone to deadly drug and food interactions and less toxic in overdose. Patients taking other MAO inhibitors must adhere to restrictive diets which require the avoidance of, inter alia, red wines, beer, aged cheese and meats, liver, yeast extracts and fava or broad beans.

Furthermore, because moclobemide's side-effect profile is so benign, it enjoys good compliance rates. Priest, R. G., 1990, Acta Psychiatr. Scand., Suppl. 360(82):39-41. Compliance is an integral component of successful treatment because the morbidity and mortality rates associated with untreated psychiatric illness are high. If a patient persistently rejects medical treatment for psychiatric illness because the initial experience in pharmacological intervention was bad, then the prognosis can be just as poor as if the patient had not been treated at all. When used in the treatment of major depression, moclobemide has proved itself an effective, gentle, patient-friendly drug.

### Therapeutic Efficacy of Moclobemide

Moclobemide, sold under the tradename AURORIX™ or MANERIX™ (F. Hoffman-La Roche, Basel, Switzerland), has been shown to be effective in the treatment of various psychiatric disorders. For example, moclobemide is marketed in Canada, Europe, Australia, New Zealand, South Africa, and Latin America as an antidepressive agent, for which it has demonstrated significant therapeutic effect in certain patient populations. Angst J. et al., 1996; Glick, I. D. et al., 1995. A review of the pharmacological properties and therapeutic use of moclobemide in depressive illness was published by Fulton and Benfield in Drugs, 52(3):450-478, 1996, updating a previous review of Fitton et al., 1992, Drugs, 43(4):561-596.

The preparation and use of moclobemide as an anti-depressant is described in U.S. Pat. No. 4,210,754 to Burkard et al. In the treatment of depression, moclobemide has been shown to have similar efficacy to tricyclic antidepressants (TCAs), selective serotonin reuptake inhibitors (SSRIs), and nonselective, irreversible MAO inhibitors. Fulton and Benfield, 1996; Fitton et al., 1992. In particular, moclobemide has been shown to be effective in treating elderly patients suffering from depression or age-related dementia. Wesnes, K. et al., 1990, Acta Psychiatr. Scand., Suppl. 360(82):71-72; U.S. Pat. No. 4,906,626 to Amrein et al.

Moclobemide has also been reported to be effective in the management of tobacco addiction (PCT publication WO 95/28934; PCT publication WO 90/04387), attention deficit disorder (Trott, G.E. et al., 1992, Psychopharmacol., 106 Suppl.:134-136), and anxiety disorders such as social phobia, obsessive-compulsive disorder and panic (U.S. Pat. No. 5,371,082 to Versiani et al.; Liebowitz, M. R. et al., 1990, Acta Psychiatr. Scand., Suppl. 360(82):29-34; Angst, J. et al., 1996, Int. Clin. Psychopharm., 11(Suppl. 3):3-7; Pollack, M. H. and Gould, R. A., 1996, Int. Clin. Psychopharm., 11(Suppl. 3):71-75).

The use of irreversible monoamine oxidase inhibitors (MAOIs) has been limited by the wide range of MAOI-drug and MAOI-food interactions that are possible, particularly with sympathomimetic medications or tyramine-containing foods, resulting in hypertensive reactions. Despite their clinical benefits, this has led to a reduction in use of such medications. Even when MAOI efficacy is documented for a given condition, many practitioners are reluctant to prescribe drugs from this class because of the risk of serious adverse reactions. This appears to be so even when MAOIs are more effective than other available treatments. Perhaps due to its classification as a monoamine oxidase inhibitor, moclobemide has not been extensively studied in the United States. In fact, moclobemide is not approved by the U.S. Food and Drug Administration. Further, there remains a great need in the field of psychiatry for additional therapies to better treat the growing number of psychiatric and psychological disorders presently described in the Diagnostic and Statistical Manual of Mental Disorders, Ed. 4, (DSM-IV), American Psychiatric Association, 1994, Washington, D.C.

### Treatment of Pain and Pain Disorders Defining Pain

One of the most significant health problems in the United States directly relates to the consequences of chronic, medically resistant pain. It is estimated that more than 1 million Americans experience cancer-related pain annually, and most do not receive effective pain relief. Ho, R. C. S., 1994, CA Cancer J. Clin., 44:259-61. Additionally, many patients with acquired immunodeficiency syndrome (AIDS) suffer from pain related to progress of the disease. The cause of this pain is varied and includes somatic, neuropathic, and idiopathic sources. Newshan G T and Wainapel S F, 1993, JANAC, 4:53-9. Pain is second to fever as the most common reason for hospitalization in AIDS patients, and there is a direct correlation between the presence of pain and length of hospital stay. Lebovits A H et al., 1989, Clin. J. Pain, 5:245-8. Persistent pain caused by cancer, sickle cell anemia, rheumatoid and auto-immune disorders, and other chronic or degenerative diseases remains a significant, unmet clinical need. Furthermore, pain can be prolonged or exacerbated by psychological factors, giving rise to “psychogenic pain disorder.” Pain has been defined by the International Association for the Study of Pain as “an unpleasant sensory and emotional experience associated with actual or potential tissue damage or described in terms of such damage”. Merskey HM, 1986, [abstract], Pain, Suppl. 3:S217. Herein, the word “pain” is used accordingly. Acute pain is defined as pain temporally related to a precipitating event. Comprehensive guidelines for acute pain management have been published by the Agency for Health Care Policy and Research and provide an excellent review of the subject (Acute Pain Management Guideline Panel, Acute pain management: operative or medical procedures and trauma Clinical practice guidelines, Rockville, MD: Agency for Health Care Policy and Research: 1992, US Dept. of Health and Human Services, Public Health Service, AHCPR publication no. 92-0032.) In relation to pain, “chronic” describes not only duration, but a syndrome with specific therapeutic implications. In addition to defining chronic pain as pain that persists for at least 3 months, the International Association for the Study of Pain also includes more than 200 clinical syndromes in the classification of chronic pain (Merskey, 1986).

### Organic Pain

For pathophysiological reasons, pain is divided into two categories: organic (having an identifiable cause) and psychogenic (lacking an organic cause). Organic pain may be nociceptive (associated with potential or ongoing tissue damage) or neuropathic (nervous system dysfunction in the absence of ongoing tissue damage).

Pain having an organic basis is generally demonstrated by a specific lesion with well-defined characteristics of pain. However, it has also been found that there are biochemical (e.g., serotonergic) abnormalities that exist without specific lesions, which are manifested by dull, diffuse pain. In the absence of a detectable, defined lesion, abnormalities at the molecular level are likely responsible for chronic pain.

Pain is a common symptom of a variety of medical and neurological diseases. Usually, this pain protects us from further injury while allowing healing to occur. It is effectively treated with various combinations of opioid and non-steroidal anti-inflammatory agents and generally resolves quickly.

Nociceptive pain involves pain arising from tissue damage that is not nerve tissue damage. Such pain, for example, may be pain derived from the presence of a tumor, from infection in an AIDS patient, from the healing of an incision in a surgical patient.

If sensory nerves have been damaged in the course of illness, or as a result of physical trauma or medical treatment (e.g., in sensory nerve damage in patients with cancer, AIDS, diabetes, or autoimmune disorders), pain often persists and is disabling and resistant to treatment.

Examples of neuropathic pain include phantom limb pain and postherpetic neuralgia. This neuropathic pain is often delayed in onset and is typically described by patients as “burning” or “shooting” in quality. Although the mechanisms underlying neuropathic pain are not known in detail, some of the determining factors have begun to be evident. These include (1) pathologic processes at the site of nerve injury (particularly inflammation), (2) abnormal excitability of peripheral sensory nerves involved in pain transmission, and (3) changes in the central nervous system that occur as a consequence of nerve injury. Davar, G., 1998, Abstract presented at the 2nd Annual Therapeutic Developments in Chronic Pain Conference, Annapolis, MD, May 18-19.

Complex Regional Pain Syndromes (CRPS) associated with neurologic dysfunction include “reflex sympathetic dystrophy” and “causalgia.” Walker, S. M., and Cousins, M. J., 1997, Anaesth. Intensive Care, 25(2):113-125. Sympathetically maintained pain is a frequent but variable component of these syndromes, as the sympathetic and somatosensory pathways are no longer functionally distinct. Pain is the cardinal feature of CRPS, but the constellation of symptoms and signs may also include sensory changes, autonomic dysfunction, trophic changes, motor impairment and psychological changes.

Diagnosis of CRPS is based on the clinical picture, with additional information regarding the presence of sympathetically maintained pain or autonomic dysfunction being provided by carefully performed and interpreted supplemental tests. Clinical experience supports early intervention with sympatholytic procedures (pharmacological or nerve block techniques), but further scientific data is required to confirm the appropriate timing and relative efficacy of different procedures.

Central neuropathic pain, or pain due to central nervous system damage, includes thalamic pain syndromes such as post-stroke thalamic pain and Dejerine-Roussy syndrome. Thalamic pain syndromes are characterized by a lesion in the thalamic area associated with intractable contralateral pain. In addition to pain, patients with Dejerine-Roussy typically experience sensory impairment, hemiparesis, ataxia, and choreoathetosis.

### Psychogenic Pain

Chronic pain is a multidimensional syndrome with both physiological and psychological contributing mechanisms. The term “psychogenic pain disorder” is used herein to describe a pain syndrome exacerbated or caused predominantly by psychological factors, in accordance with the DSM-IV. According to the DSM-IV, pain associated with a general medical condition alone, absent psychological contributing factors, is a purely physical syndrome that should not be characterized as a mental disorder. Accordingly, the term “psychogenic pain disorder” is used herein to denote pain with a clinically significant psychological aspect.

Psychogenic Pain Disorder can be associated either with psychological factors alone, or with both psychological and physiological factors. Acute psychogenic pain disorder is characterized by pain lasting less than 6 months, while chronic psychogenic pain disorder describes pain longer than 6 months in duration.

The essential feature of psychogenic pain disorder is pain that is the predominant focus of the clinical presentation and is of sufficient severity to warrant clinical attention. The pain causes significant distress or impairment in social, occupational, or other important areas of function. Psychological factors are thought to play a significant role in the onset, severity, exacerbation, or maintenance of the pain.

### Psychosocial Components of Pain

The social, emotional, and economic dysfunction associated with chronic pain and psychogenic pain disorder typically results in an inability to work or attend school, frequent use of the health care system, substantial use of medications, and relational problems such as marital discord and disruption of normal social and familial relationships. The pain becomes the primary focus of the patient's life, and significant time and resources are expended trying to locate the “cure.”

Perhaps due to the high incidence of unemployment, disability, and family problems associated with chronic forms of pain and psychogenic pain disorder, substance abuse is frequently encountered among the patient population. Patients with organic and psychogenic chronic pain syndromes tend towards inactivity and social isolation, which frequently leads to additional psychological and physical problems. Indeed, individuals whose chronic pain is associated with terminal illness, most notably cancer, appear to be at an increased risk for suicide. DSM-IV.

Despite the development of new instruments and treatments to assess and manage organic and psychogenic pain disorders, chronic pain is often poorly understood and inadequately addressed. Garcia, J., and Altman, R. D., 1997, Semin. Arthritis Rheum., 27(1):1-16. Traditionally, drug therapy has relied on the nonsteroidal anti-inflammatory drugs (NSAIDs) and opioid analgesics for chronic nociceptive pain.

Methadone is a synthetic opioid agonist considered a preferred drug in the management of pain. Ripamonti, C. et al., 1997, Pain, 70(2-3):109-115. Methadone has a number of unique characteristics including excellent oral and rectal absorption, no known active metabolites, high potency, low cost, and longer administration intervals, as well as an incomplete cross-tolerance with respect to other mu-opioid receptor agonist drugs. For these reasons, methadone has the potential of playing a major role in the treatment of pain, particularly cancer pain.

However, the use of methadone is limited by the remarkably long and unpredictable half-life, large inter-individual variations in pharmacokinetics, the potential for delayed toxicity, and above all by the limited knowledge of correct administration intervals and the equi-analgesic ratio with other opioids when administered chronically. Future research is needed to better define the variation in both bioavailability and elimination of methadone in different patient populations, the interaction between methadone and the most commonly used drugs in cancer patients, the type and activity of potential methadone metabolites, and the equi-analgesic doses between methadone and the most commonly used opioids. Ripamonti et al., 1997.

A newer analgesic choice for moderate to moderately severe pain is tramadol, a centrally acting agent with at least two complementary mechanisms of action and minimal gastrointestinal or renal toxicity. Aronson, M. D., 1997, Clin. Ther., 19(3):420-432. Tramadol works through a combined mechanism of weak mu receptor binding and the inhibition of serotonin and norepinephrine reuptake. Tramadol has a favorable adverse-effect profile and therefore is likely to have an important role in the management of chronic pain syndromes. However, the efficacy of such compounds in treating psychogenic pain disorders remains unknown.

Adjuvant agents, including tricyclic antidepressants (TCAs), anticonvulsants, and local anesthetics, also help manage chronic neuropathic pain. Garcia and Altman, 1997. TCAs have been reported to be valuable in several specific pain disorders, including low-back pain, fibromyalgia, postherpetic neuralgia, and neuropathic pain. Magni G., 1991, Drugs, 42:730-48. Three other anti-depressants, namely desipramine, fluvoxamine, and moclobemide, have been shown to have an antinociceptive effect after single oral dosing in a randomized, double-blind, placebo-controlled crossover study in 10 healthy volunteers exposed to an acute nociceptive stimulus. Coquoz, D. et al., 1993, Clin. Pharmacaol. Ther., 54(3):339-344. Again, however, the efficacy of such compounds in treating or preventing either chronic organic pain or psychogenic pain disorder is, as yet, untested.

Although significant advances in the understanding of chronic pain and its pathophysiological mechanisms and newer techniques (noninvasive and invasive) for organic chronic pain management have become available, reduced patient morbidity and improved quality of life may only be realized with an improved understanding of available resources. Moreover, the area of psychogenic pain disorder remains only poorly understood, and more effective treatment modalities are needed. Pain and its management remains a challenge for all medical sub-specialties. Despite the wide range of drugs available, opioids remain the major therapy for moderate to severe pain, and pharmaceutical interventions for psychogenic pain disorder have not been developed to any greater degree. Due to the limitations of available pharmaceutical interventions, alternate pain treatment options with greater efficacy, lower likelihood of inducing dependence, and fewer adverse side-effects are needed.

### Treatment of Posttraumatic Stress Disorder

Posttraumatic Stress Disorder (PTSD) is a syndrome characterized by clinically significant distress that results in disabling social and occupational dysfunction for periods of more than one month. American Psychiatric Association, Diagnostic and Statistical Manual of Mental Disorders, Ed. 4, Washington, D.C., American Psychiatric Association, 1994, pp. 424-429 (DSM-IV). The essential feature of PTSD is the development of characteristic symptoms following exposure to an extreme traumatic stressor involving direct personal experience of an event that involves actual or threatened death or serious injury, or other threat to one's physical integrity; or witnessing an event that involves death, injury, or a threat to the physical integrity of another person; or learning about the unexpected or violent death, serious injury, or threat of death or injury experienced by a family member or other close associate.

The patient's response to the event generally involves disorganized or agitated behavior. The characteristic symptoms resulting from the exposure to the extreme trauma include persistent re-experience of the traumatic event, persistent avoidance of stimuli associated with the trauma and numbing of general responsiveness, as well as persistent symptoms of increased arousal and anxiety not exhibited by the patient before the traumatic incident.

The traumatic event is most commonly re-experienced by way of recurrent and intrusive recollections of the events or recurrent distressing dreams of the event. Intense psychological distress or physiological reactivity often occurs when the person is exposed to triggering events that resemble or symbolize an aspect of the traumatic event. Thus, stimuli associated with the event and persistently avoided by patients suffering from PTSD. The patient typically makes deliberate efforts to avoid thoughts, feelings, or conversations about the event and avoids activities, situations, or people who are associated with the event.

PTSD can manifest itself chronically, defined as the presence of the full complement of symptoms for a period of 3 months or longer, or it may appear acutely, with each episode lasting less than 3 months. Occasionally, delayed-onset PTSD occurs, wherein at least 6 months have passed between the traumatic event and the onset of the above-described symptoms.

Beyond its medical implications, PTSD presents a serious threat to the social and economic well-being of its victims. PTSD is a disabling disease affecting 1% -14% of people in the United States. Studies of high risk groups, such as combat veterans and victims of natural disasters or violent crime, have yielded prevalence rates ranging from 3% to 58%. Individuals with PTSD frequently experience problematic interpersonal relationships, leading to marital conflict, loss of employment, and withdrawal from society in general. Furthermore, patients suffering from PTSD are at an increased risk for other debilitating psychiatric disorders, including Panic Disorder, Agoraphobia, Obsessive-Compulsive Disorder, Social Phobia, Major Depressive Disorder, and Substance Abuse. PTSD is also frequently associated with self-destructive, self-mutilation, and impulsive behavior resulting in injury to the patient, such as head trauma or burns.

Conventional treatment for PTSD has focused mainly on psychological therapy. More recently, select compounds with anti-depressant activity have shown moderate promise in the prevention and treatment of PTSD. For example, in an open trial with 5 patients suffering from traumatic war neuroses responded positively to phenelzine after failing to benefit from antipsychotics, tricyclics, and psychotherapy. Hogben, G. L., and Cornfield, R. B., 1981, Arch. Gen. Psychiatry, 48:440-445. Davidson et al. (1987, Br. J. Psychiatry, 150:252-255) also found phenelzine helpful in 8 of 11 PTSD patients in an open trial, and phenelzine has been shown superior to placebo in reducing PTSD symptoms. Frank, J. B. et al., 1988, Am. J. Psychiatry, 145:1289-1291. However, others have reported only modest clinical improvement in PTSD patients treated openly with phenelzine for a period of 4 to 18 weeks. Lerer, B. et al., 1987, Arch. Gen. Psychiatry, 44:976-981. Moreover, roughly 25% of potential treatment candidates in the Frank et al. study refused to participate due, in part, to reluctance to take medication with side effects such as sedation.

Due to the limitations of available psychotherapy and pharmaceutical interventions, alternate PTSD treatment options with greater efficacy and fewer adverse side-effects are presently being sought. Liebowitz, M. R. et al., 1990, Acta Psychiatr. Scand., Suppl. 360(82):29-34.

### Premenstrual Dysphoric Disorder and Premenstrual Syndrome

Premenstrual dysphoric disorder (PMDD) was first mentioned as a special psychiatric diagnosis in the Diagnostic and Statistical Manual of Mental Disorders (DSM-IV) in 1994. Prior to then, it was referred to as late luteal phase dysphoric disorder DSM-III-R-Appendix A.

The essential features of PMDD are symptoms such as markedly depressed mood, marked anxiety, marked affective lability, and decreased interest in activities. These symptoms usually occur regularly during the last week of the luteal phase in most menstrual cycles during the year. The symptoms typically begin to remit within a few days of the onset of menses (the follicular phase), and are always absent during the week following menses.

According to the DSM-IV, the presence of five or more of the following symptoms during the last week of the luteal phase, with at least one of the symptoms being among the first four listed, is indicative of clinical PMDD: 1) feeling sad, hopeless, or self-deprecating; 2) feeling tense, anxious, or “on edge”; 3) marked lability of mood interspersed with frequent tearfulness; 4) persistent irritability, anger, and increased interpersonal conflict; 5) decreased interest in usual activities, which may be associated with withdrawal from social relationships; 6) difficulty concentrating; 7) feeling fatigued, lethargic, or lacking energy; 8) marked changes in appetite, which may be associated with binge eating or craving certain foods; 9) hypersomnia or insomnia; 10) a subjective feeling of being overwhelmed or out of control; and 11) physical symptoms such as breast tenderness or swelling, headaches, or sensations of bloating or weight gain, with tightness of fit of clothing, shoes, and rings. There may also be joint or muscle pain. In more severe cases, the symptoms may be accompanied by suicidal thoughts.

The pattern of symptoms typically occurs most months, and disappears shortly after the onset of menstruation. The most typical pattern appears to be that of dysfunction during the week prior to menses, ending mid-menses. Atypically, some females also have symptoms for a few days during ovulation. As a result, females with particularly short menstrual cycles may be symptom-free for as little as one week per cycle.

The duration and persistence of PMDD is particularly troubling given its severity; the symptoms of PMDD are of comparable severity to those of a Major Depressive Episode (DSM-IV) and frequently cause an obvious and marked impairment in the ability to function socially or professionally. Impairment in social function may be manifested by marital discord and difficulty interacting with friends and family. There is generally a distinct contrast between mood, capabilities, and function, during the week preceding menses, and her mood and capabilities during the remainder of the month.

Although there are no specific laboratory tests that are diagnostic of PMDD, several small preliminary studies indicate that certain laboratory findings, such as serotonin or melatonin secretion patterns and sleep EEG findings, may be abnormal in females thought to suffer from PMDD. (DSM-IV).

In addition to PMDD, premenstrual syndrome (PMS), a disorder associated with less severe anxiety, depression, and mood swings, is relatively common among menstruating females. Criteria differentiating PMDD from PMS are the requirements that PMDD patients, unlike those with PMS, have at least five of the symptoms listed in the DSM-IV, including one mood symptom; have impairment associated with the illness; and prospectively confirm the symptoms.

Like PMDD, PMS occurs in the second half of the menstrual cycle, when brain levels of progesterone and its metabolites decline. Recent studies suggest that PMS may be due to a change in the makeup of the receptor for the inhibitory amino acid receptor γ-aminobutyric acid (GABA). (Smith et al., 1998, Nature, 392:926). The study explains why benzodiazepine antianxiety drugs have been ineffective in treating PMS-related anxiety.

It is estimated that at least 75% of women report minor or isolated premenstrual changes in mood and function. Limited studies suggest an occurrence of premenstrual syndrome (PMS) in 20%-50% of women, and that 3%-5% experience symptoms that meet the criteria for PMDD. Gehlert, S. and Hartlage, S., 1997, J. Psychosom. Obstet. Gynaecol., 18(1):36-44.

Treatment options range from the conservative for less severe PMS (lifestyle and stress management) to treatment with psychotropic medications and hormonal or surgical interventions to eliminate ovulation for the more extreme cases. Results from several randomized, placebo-controlled trials have clearly demonstrated that selective serotonin reuptake inhibitors, as well as medical or surgical oophorectomy, are effective in treating premenstrual dysphoric disorder. Taken together, these data indicate that treatment may be accomplished by either eliminating the hormonal trigger or by reversing the sensitivity of the serotonergic system. Steiner, M., 1997, Annu. Rev. Med., 48:447-55.

Such studies have led to speculation that premenstrual disorders such as PMDD and PMS are the end result of a complex series of events mediated partly by the serotonin system and triggered by ovulation. Korzekwa, M. I. and Steiner, M., 1997, Clin. Obstet. Gynecol., 40(3):564-76; Halbreich, U., 1997, Acta. Psychiatr. Scand., 95(3):169-76. Accordingly, the most consistent positive results in treating PMDD or PMS have been found for compounds active at serotonin receptors, such as selective serotonin reuptake inhibitors (SSRIs). Yonkers, K. A., 1997, J. Clin. Psychiatry, 58 Suppl. 14:4-10; discussion 11-3. For example, the SSRI sertraline was found to be significantly better than placebo for treatment of PMDD as reflected by symptomatic improvement and change in reported functional impairment. Yonkers, K. A. et al., 1997, JAMA, 278(12):983-8; Cohen, L.S., 1998, JAMA, 279(5):357-8. Similarly, fluoxetine has been shown to be an effective and well-tolerated drug with considerable promise in treating a range of symptoms in women with PMS. Ozeren, S., et al., 1997, Eur. J. Obstet. Gynecol. Reprod. Biol., 73(2):167-70; Su, T. P. et al., 1997, Neuropsychopharmacology, 16(5):346-56.

However, the effects of selective serotonin reuptake inhibitors on menstrual cycle length are mostly unknown and warrant careful monitoring in women of reproductive age. Steiner, M. et al., 1997, Obstet. Gynecol., 90 (4 Pt 1):590-5. Furthermore, studies indicate that some common adverse side effects of treatment with serotonin reuptake inhibitors, such as sexual dysfunction (reported by 8.5% of patients treated, Ozeren et al., 1997), persist unchanged for at least 10 consecutive cycles of treatment. Sundblad, C. et al., 1997, Eur. Neuropsychopharmacol., 7(3):201-6.

The efficacy of nonserotonergic antidepressants is less well studied. In one study comparing the efficacy of fluoxetine (an SSRI), bupropion (a non-SSRI), and placebo in women with PMDD, efficacy and patient satisfaction were both greater with fluoxetine, although some improvement with bupropion was noted, and both medications were well tolerated. Pearlstein, T. B. et al., 1997, J. Clin. Psychopharmacol., 17(4):261-6. Recently, randomized controlled clinical trials of treatments for clearly diagnosed PMS and/or PMDD reported efficacy for a gonadotropin-releasing hormone agonist (Freeman, E. W. et al., 1997, Psychopharmacol. Bull., 33(2):303-9), and preliminary data suggested efficacy for spironolactone and a carbohydrate-rich beverage. Freeman, E. W., 1997, Curr. Opin. Obstet. Gynecol. 9(3):147-53.

Due to the pervasive, chronic, and serious nature of PMDD and PMS, and the persistent adverse side effects associated with the most common pharmacological interventions, there remains a need to further refine methods to treat premenstrual syndromes.

### Sleep Disorders

As characterized by the DSM-IV, sleep disorders fall into four major categories based on their respective etiologies. (DSM-IV, pp. 551-607; See also The International Classification of Sleep Disorders: (ICSD) Diagnostic and Coding Manual, 1990, American Sleep Disorders Association.) One category, Primary Sleep Disorders, comprises sleep disorders that do not result from another mental disorder, a substance, or a general medical condition.

A second category comprises those sleep disorders attributable to substances, including medications and drugs of abuse. A third category comprises sleep disturbances arising from the effects of a general medical condition on the sleep/wake system. A fourth category of sleep disorders comprises those resulting from an identifiable mental disorder such as a mood or anxiety disorder.

### Primary Sleep Disorders

Primary Sleep Disorders are subdivided into (a) Dyssomnias—disorders of initiating or maintaining sleep, or of excessive sleepiness, characterized by abnormalities in the amount, timing, or quality of sleep; and (b) Parasomnias—disorders characterized by abnormal behavioral or physiological events associated with sleep, particular sleep stages, or sleep/wake transitions.

Dyssomnias include, for example, Primary Insomnia, Primary Hypersomnia, Narcolepsy and Circadian Rhythm Sleep Disorder.

Primary Insomnia is characterized by an inability to initiate or maintain sleep, or by nonrestorative sleep, persists for at least one month and that significantly interferes with social, occupational, or other functioning. Affected individuals typically experience a combination of difficulty falling asleep and intermittent wakefulness. Less commonly, affected individuals may complain only of nonrestorative sleep, that is, feeling that their sleep was restless, light or of poor quality. Primary Insomnia is often associated with increased physiological or psychological arousal at nighttime in combination with negative conditioning for sleep. A marked preoccupation with and distress due to the inability to sleep may contribute to the individual's difficulty in sleeping, causing the individual to become more frustrated and distressed. Conversely, the affected individual may fall asleep more easily when not trying to do so, e.g., while relaxing away from the bedroom. Chronic primary insomnia may lead to deterioration of mood and motivation, decreased attention, general malaise, and fatigue. Although individuals often complain of daytime fatigue, polysomnographic studies usually do not demonstrate an increase in physiological signs of sleepiness.

Many individuals with primary insomnia have a history of “light” or easily disturbed sleep prior to the development of more persistent sleep problems or problems meeting a clinically significant threshold of intensity or duration. Other associated factors may include anxious overconcern with general health and increased sensitivity to the daytime effects of mild sleep loss.

Primary insomnia typically begins in young adulthood or middle age and is rare in children or adolescents. In exceptional cases, the insomnia can be traced back to childhood. The course of primary insomnia is variable. It may be limited to a period of several months, particularly if precipitated by a psychosocial or medical stressor that later resolves. The more typical course consists of an initial phase of progressive worsening over weeks to months, followed by a chronic phase of stable sleep difficulty that may last for many years. Some individuals experience an episodic course, with periods of better or worse sleep occurring in response to life events such as vacations or stress.

The true prevalence of primary insomnia in the general population is unknown, although population surveys indicated a 1-year prevalence of insomnia complaints in 30%-40% of adults. The percentage of those whose sleep disturbance would meet criteria for primary insomnia has not been studied. Primary insomnia appears to occur more frequently with increasing age, and among women.

Primary insomnia subsumes a number of insomnia diagnoses in the International Classification of Sleep Disorders (ICSD), including Psychophysiological Insomnia, Sleep State Misperception, Idiopathic Insomnia, and some cases of Inadequate Sleep Hygiene. Psychophysiological Insomnia most closely resembles Primary Insomnia, particularly in terms of arousal and conditioning factors. Sleep State Misperception is a condition characterized by complaints of insomnia with a marked discrepancy between subjective and objective estimates of sleep. Idiopathic Insomnia includes those cases with onset in childhood and a lifelong course, presumably due to an abnormality in the neurological control of the sleep-wake system. Inadequate Sleep Hygiene refers to insomnia resulting from behavioral practices that increase arousal or disrupt sleep organization (e.g., working late into the night, taking excessive daytime naps, or keeping irregular sleep hours).

Primary Hypersomnia is characterized by excessive sleepiness of at least one month's duration, evidenced by near-daily daytime sleep episodes, excessive daytime naps, or prolonged sleep episodes. Because the actual quality of nocturnal sleep is normal, persons suffering from primary hypersomnia sleep efficiently, but they do not wake refreshed, and may display signs of “sleep drunkenness,” or difficulty making the transition from sleep to wakefulness. Daytime naps tend to be relatively long (often lasting an hour or more), are experienced as unrefreshing, and often do not lead to improved alertness. Affected individuals typically feel sleepiness developing over a period of time, rather than experiencing a sudden sleep “attack.” Unintentional sleep episodes typically occur in low-stimulation and low-activity situations (e.g., while attending lectures, reading, watching television, or driving long distances).

In its clinical manifestation, primary hypersomnia is severe enough to cause significant distress or substantial disruptions of social, occupational, or interpersonal functions. In particular, the low level of daytime alertness may interfere with the affected individual's ability to work or carry on normal activities.

Primary hypersomnia typically begins between ages 15 and 30, thereafter becoming chronic. Most individuals with primary hypersomnia exhibit consistent and persistent symptoms. In contrast, some patients experience symptoms periodically, in episodes of several days to several weeks, with symptomatic periods recurring several times per year. Between periods of excessive sleepiness, sleep duration and daytime alertness are normal.

A subset of individuals with Primary Hypersomnia have a family history of hypersomnia and also have symptoms of autonomic nervous system dysfunction, including recurrent vascular-type headaches, reactivity of the peripheral vascular system (Raynaud's phenomenon), and fainting.

The true prevalence of primary hypersomnia in the general population has not been established, although roughly 5%-10% of sleep disorder patients are thought to be affected.

Primary hypersomnia is analogous to the diagnosis of idiopathic hypersomnia in the ICDS. In addition, the ICDS includes a separate category for recurrent hypersomnia, which is analogous to the recurrent form of primary hypersomnia.

Narcolepsy is a sleep disorder characterized by repeated irresistible episodes of refreshing sleep, cataplexy (the sudden reversible loss of muscle tone), and intrusions of elements of rapid eye movement (REM) sleep into the transition period between sleep and wakefulness manifested by paralysis of voluntary muscles or dreamlike hallucinations. The essential feature of narcolepsy is the sudden, unintentional onset of sleep episodes in inappropriate situations, such as while operating a motor vehicle or carrying on a conversation, that occur daily over a period of at least 3 months. Each sleep episode typically lasts approximately 10-20 minutes, and untreated affected individuals may have 2-6 episodes of sleep per day, including intentional sleep. Episodes of sleepiness in Narcolepsy are often described as irresistible, and individuals have varying abilities to “fight off” these sleep attacks.

Many sleep experts allow the diagnosis to be made in the absence of cataplexy or intrusions of REM sleep elements if the individual demonstrates pathological sleepiness and two or more sleep-onset REM periods during a Multiple Sleep Latency Test (MSLT).

Cataplexy often develops several years after the onset of daytime sleepiness and occurs in approximately 70% of individuals with the disorder. The loss of muscle tone with cataplexy may be subtle, such as a sagging jaw or drooping eyelids, head, or arms that may not be noticeable to observers, or it may be more severe, resulting in an inability to maintain an upright position or carry objects.

Respiratory and eye muscles are generally not affected. The muscle weakness usually lasts only seconds, although periods of up to half an hour have been reported. Episodes are followed by a full return of normal muscle strength. Full consciousness and alertness are preserved during cataplectic episodes. Individuals can clearly describe events and have no confusion before or after the episode. Rarely, prolonged episodes of cataplexy may lead into sleep episodes. Cataplexy is usually triggered by a strong emotional stimulus (e.g., anger, surprise, laughter). Sleep deprivation typically increases the frequency and severity of episodes of cataplexy.

Approximately 20%-40% of affected individuals additionally experience intense, sometimes frightening hallucinations just prior to falling asleep or just after awakening. Most sleep-related hallucinations are visual and incorporate elements of the actual environment. The hallucinations may also be auditory (e.g., hearing intruders in the home) or kinetic (e.g., sensation of flying).

The onset of narcolepsy generally occurs in adolescence, with cataplexy sometimes not appearing until months or years after the initial Narcoleptic episodes.

Approximately 30%-50% of individuals with Narcolepsy also experience sleep paralysis just on falling asleep or awakening. In this condition, individuals describe being awake but unable to move or speak. They may also complain of feeling unable to breathe, although the diaphragm is spared and respiration continues. Sleep-related hallucinations and sleep paralysis may occur simultaneously, resulting in an often terrifying experience of seeing or hearing unusual things and being unable to move. Both sleep-related hallucinations and sleep paralysis last for seconds to a few minutes and terminate spontaneously. Both phenomena are thought to result from dissociated elements of REM sleep intruding into wakefulness. Narcolepsy appears to have a stable course over time, and epidemiological studies indicate that narcolepsy affects 0.02%-0.16% of the population, and affects both sexes equally.

Data from family studies strongly suggests a role for genetic factors in the development of narcolepsy. The mode of inheritance has not been determined but is likely multifactorial. Approximately 5%-15% of first-degree biological relatives of probands with narcolepsy have the disorder. Approximately 25%-50% of the first-degree biological relatives of individuals with narcolepsy have other disorders characterized by excessive sleepiness (such as primary hypersomnia).

Narcolepsy is classified in the chapter of ICSD devoted to neurological conditions.

Circadian Rhythm Sleep Disorder (CRSD) is a recurrent of persistent pattern of sleep disruption characterized by a mismatch between the individual's normal sleep/wake cycles and the timing and duration of periods available for sleep. In contrast to other primary Sleep Disorders, CRSD does not result from the mechanisms generating sleep and wakefulness per se. As a result of this circadian mismatch, affected individuals may complain of insomnia at certain times of the day and excessive sleepiness at other times, with such periods occurring in a manner that interferes with that individual's social, economic, and interpersonal well-being.

Individuals displaying Delayed Sleep Phase Type CRSD are unable to modify their sleep cycle by sleeping at an earlier time, while individuals displaying Advanced Sleep Phase Type CRSD are unable to delay sleep to a later, more appropriate time. Affected individuals often experience impairment of social, occupational, and interpersonal functions.

In jet-lag type CRSD, the endogenous circadian sleep-wake cycle is normal and the disturbance arises from conflict between the pattern of sleep and wakefulness generated by the circadian system and the pattern of sleep and wakefulness required by a new time zone. Individuals with this type complain of a mismatch between desired and required hours of sleep and wakefulness. Eastward travel (advancing sleep-wake hours) is typically more difficult for most individuals to tolerate than westward travel (delaying sleep-wake hours). Without intervention, CRSD may persist for years or decades.

Unlike dyssomnias, such as those described above, parasomnias do not involve abnormalities of the mechanisms generating states, nor of the timing of sleep. Rather, parasomnias are characterized by inappropriate physiological activity during sleep. In particular, the autonomic nervous system, motor system, and cognitive processes appear to be disturbed in parasomniacs. Individuals with parasomnias usually present with complaints of unusual behavior during sleep, rather than complaints of insomnia or excessive daytime sleepiness. Parasomnias may include, for example, nightmare disorder, sleep terror, and sleepwalking disorder.

Nightmare disorder (formerly referred to as dream anxiety disorder) is manifested by repeated occurrences of terrifying nightmares, from which the individual awakens fully alert and with significant recollection of the nightmare. Affected individuals may awaken several times during a night, or may begin to avoid sleep to avoid the nightmares. Nightmare disorder can lead to social, interpersonal, and occupational impairment. However, the affected individual more often experiences significant subjective distress.

Nightmares typically occur in a lengthy, elaborate dream sequence that is highly anxiety provoking or terrifying. Dream content most often focuses on imminent physical danger to the individual (e.g., pursuit, attack, injury). Nightmares that occur after traumatic experiences may replicate the original dangerous or threatening situation, but most nightmares do not recount actual events.

On awakening, individuals with this disorder can describe the dream sequence and content in detail. Individuals may report multiple nightmares within a given night, often with a recurrent theme. Nightmares arise almost exclusively during rapid eye movement (REM) sleep. Because REM episodes occur periodically throughout nocturnal sleep (approximately every 90-110 minutes), nightmares may also occur at any time during the sleep episode. However, because REM sleep periods typically become longer and dreaming more intense in the second half of the night, nightmares are also more likely to occur late in the night.

Nightmare disorder is seen most commonly in childhood, and females experience the disorder approximately four times more than males. Nightmare disorder corresponds to the diagnosis of Nightmares in the ICSD.

A similar disorder, Sleep Terror Disorder, is characterized by the repeated occurrence of sleep terrors—abrupt awakenings from sleep that usually are marked by a panicky scream or cry. Sleep terror disorder can be differentiated from nightmare disorder in that the affected individual does not remember the terrifying dream, and the events typically occur earlier in the night than in Nightmare Disorder. The episodes are marked by autonomic arousal and behavioral manifestations of intense fear. During an episode, the individual is difficult to awaken or comfort. If the individual awakens after the sleep terror, no dream is generally recalled, or only fragmentary, single images are recalled. On awakening the following morning, the individual typically has no memory of the event. Sleep terrors are also called “night terrors” or pavor nocturnus.

Episodes of sleep terror disorder generally last from 1-10 minutes. Each episode is usually accompanied by yelling, screaming, crying, or incoherent vocalizations. The individual may actively resist being held or touched, or even demonstrate more elaborate motor activity. Sleep terror disorder causes clinically significant impairment of social, occupational, or other functioning.

Statistics of the disorder's prevalence are limited, but the disorder appears to afflict children more often than adults. In children, it typically appears between ages 4 and 12, and generally resolves during adolescence. In adults, the disorder usually manifests itself between ages 20 and 30, and generally follows a chronic course.

Individuals with sleep terror disorder frequently report a positive family history of either sleep terrors or sleepwalking. Some studies indicate a tenfold increase in the prevalence of the disorder among first-degree biological relatives. The exact mode of inheritance is unknown.

Sleepwalking disorder is characterized by repeated episodes of behavior involving complex motor activity initiated while the affected individual is asleep. Motor activity is most often initiated within the first third of the night. Sleepwalking episodes can include a variety of behaviors. In mild episodes, sometimes called “confusional arousal”, the individual may only sit up or look around. More typically, the individual actually gets out of bed and may walk around. Sleepwalking individuals may stare blankly and are generally unresponsive to efforts by others to communicate with or awaken them. If awakened during a sleepwalking episode, the individual typically has limited recall of the event. Immediately following the episode, the individual may be confused or disoriented, but generally recovers cognitive function quickly.

Affected individuals experience impairment of social, occupational, or interpersonal functioning as a result of the sleepwalking. Onset of sleepwalking disorder occurs most frequently between ages 4 and 8 years old, generally with spontaneous disappearance during early adolescence. Approximately 1%-5% of children are affected by Sleepwalking disorder. Sleepwalking disorder is virtually identical to Sleepwalking as described in ICSD. The ICSD includes two other disorders that may have features similar to sleepwalking: Confusional Arousals and Nocturnal Eating (Drinking) Syndrome.

Other Primary Sleep Disorders of interest include, without limitation, REM Sleep Behavior Disorder (RSBD), in which the affected individual displays excessive motor activity during sleep, and Sleep Paralysis.

### Substance-Induced Sleep Disorder

Substance-induced sleep disorder is characterized by a clinically-significant disturbance of sleep that is the direct result of the intake of a substance, including drugs of dependence or abuse, medications, or toxins. Affected individuals may most commonly experience insomnia or hypersomnia, but parasomnias have also been noted, as have mixed-type sleep disorders.

### Sleep Disorder due to a General Medical Condition

Sleep Disorder Due to a general medical condition is characterized by a prominent and severe disturbance in sleep warranting independent clinical attention. The disorder may include insomnia, hypersomnia, parasomnias, or any combination thereof.

In determining whether the sleep disturbance is due to a general medical condition, the clinician must first establish the presence of a general medical condition. Further, the clinician must establish that the sleep disturbance is etiologically related to the general medical condition through a physiological mechanism. Although there are no precise guidelines for determining whether the sleep disturbance and the general medical condition are etiologically related, factors such as (1) the presence of a temporal association between the onset, exacerbation, or remission of the general medical condition and that of the sleep disturbance and (2) the presence of features that are atypical of primary Sleep Disorders should be considered.

Existing pharmaceutical therapies for sleep disorders vary depending upon the particular disorder. Narcolepsy is generally treated with stimulants, TCAs that act as REM-suppressants, and scheduled naps. Tolerance to prescribed stimulants, however, can occur. Lee, K. A., 1997, ANNA J., 24(6):614-23, 677. TCAs achieving REM-suppression can also help persons with Circadian Rhythm Disorder. Id. Melatonin has been shown to promote sleep when provided exogenously; one study of young healthy adults demonstrated that a 5 mg dose increased sleep propensity and the duration of sleep. Zhdanova, I. A., Lynch, H. J., and Wurtman, R. J., 1997, Sleep, 20(10):899-907. Such studies indicate that melatonin has potential as a treatment for disorders involving the circadian rhythm shifts and insomnia, but its potential for toxicity and other harmful effects is only poorly characterized. Id. Individuals with RSBD are usually treated with clonazepam, which is effective in approximately 90% of cases. Such individuals also respond to REM-suppressing TCAs; however, the use of such medications poses risks in the elderly. Moreover, at least one study indicates that TCAs may actually induce transient RSBD. Chiu, H. F. K. and Wing, Y. K., 1997, Intl J. Clin. Pract. 51(7):451-54. L-dopa or bromocriptine may also help, but patients develop nausea and tolerance to the drugs, rendering them unfeasible. Lee, K. A., 1997, ANNA J. 24(6):614-23, 677.

Due to the limitations of available pharmaceutical interventions for sleep disorders, there remains a need for an effective treatment with a positive safety profile and fewer adverse side effects. To the best of applicant's knowledge, moclobemide has never been used in the treatment of sleep disorders.

Eating Disorders

Eating disorders are clinically significant disturbances in eating behavior. Some eating disorders such as bulimia nervosa and anorexia nervosa have been officially classified in the DSM-IV. There are some patients that experience eating disorders such as carbohydrate craving, obesity, overeating or binge-eating/purging or restrictive eating that do not fit the criteria of an eating disorder according to the DSM-IV. The term carbohydrate craving refers to a clinically significant tendency to frequently crave carbohydrate-containing foods (particularly, carbohydrate-rich containing snack foods such as potato chips, pastries, cookies), which is well known in the art (e.g., Wurtman, R. J. et al., Obes. Res. Suppl 4:477S-480S (1995). Patients suffering from this tendancy do not necessarily suffer from obesity.

Bulimia nervosa, or bulimia, is a disorder described in the DSM-IV that is characterized in part by recurrent episodes of binge eating during which the patient experiences a loss of control over eating and engages in self-induced vomiting. Bulimia may occur in either a purging or a non-purging subtype. The disorder primarily afflicts females of upper and middle socioeconomic status, especially college-age women. A related syndrome, binge eating disorder, has been provisionally categorized by the DSM-IV as a disorder distinct from bulimia in that the affected individual does not regularly indulge in inappropriate compensatory behaviors, such as purging, fasting, or excessive exercise.

Anorexia Nervosa, or anorexia, is a disorder marked by the refusal to maintain a minimally normal body weight, intense fear of weight gain, and a significant disturbance in the perception of body shape and/or size. Anorexia occurs in either a restricting (i.e., marked by dieting, fasting, or excessive exercise) or a binge-purge subtype. DSM-IV. There is an increased risk of anorexia among first-degree biological relatives of individuals with the disorder, and studies in twins have found a significantly higher concordance rate for monozygotic twins than for dizygotic twins. DSM-IV.

Currently, two approaches for treating bulimia and other eating disorders are commonly used: cognitive-behavioral therapy (CBT) and pharmacologic intervention. Numerous controlled treatment trials have shown CBT to be at least as efficacious as any other treatment for bulimia to which it has been compared. Wilfley, D. E. and Cohen, L. R., 1997. Interpersonal therapy, and the combination of CBT with medication are both promising treatment alternatives to CBT alone.

Traditionally, pharmacologic therapy for bulimia has involved the administration of antidepressants. Longer-term treatment of bulimia and BED with antidepressant medication, either alone or in combination with psychotherapy, are fairly promising. The use of a single antidepressant agent has been reported to result in recovery of about 25 percent of patients entering treatment; continued treatment is reportedly accompanied by relapse in about one-third of these patients. Substituting one or more antidepressants for the initial agent in patients who fail to improve or cannot tolerate side effects is thought to improve long-term maintenance. Instituting CBT may prevent relapse once medication is discontinued, and the combination of CBT and antidepressant treatment may be more effective than a single medication. There is also evidence that antidepressant treatment combined with CBT is more effective than placebo plus CBT. Agras W S, 1997, Psychopharmacol Bull, 33(3):433-6. Thus, one study has demonstrated that the combination of pharmacotherapy (treatment with the antidepressant fluoxetine) and psychotherapy was superior to pharmacotherapy alone on specific parameters, and there was no statistically significant advantage to the combination over psychotherapy alone. Goldbloom, D. S. et al., 1997, Behav. Res. Ther., 35(9):803-11.

More recent research into the fundamental causes of bulimia, however, has suggested other pharmacologic treatments. In particular, the extent to which dysregulation of serotonin function in the central nervous system may contribute to core symptoms in patients with bulimia nervosa and anorexia nervosa is currently an area of intensive psychobiological investigation. Preclinical and clinical studies have demonstrated the involvement of the neurotransmitter serotonin in the regulation of food intake, suggesting that impaired serotonin-mediated satiety signals could contribute to patters of recurrent binge eating. Other symptom patterns in patients with eating disorders, including mood dysregulation, impulsivity, and obsessionality, as well as therapeutic response to serotonergic agents, suggest involvement of serotonergic pathways. Wolfe B. E.; Metzger E.; Jimerson D. C., 1997, Psychopharmacol Bull, 33(3):345-54.

Some researchers have argued that the pathophysiological characteristics driving the behaviors characteristic of bulimia involve an increase in the basal tone of the vagal nerve, and have suggested that compounds such as ondansetron may be useful for the treatment of bulimia. Faris, P. L. et al., Biol Psychiatry 32:462-466 (1992); Dumuis et al., N. S. Arch. Pharmacol., 340:403-410 (1989).

As a class, benzamide derivatives have several prominent pharmacological actions due to their effects on neuronal systems modulated by the neurotransmitter serotonin.

Because of their modulation of the serotonin neuronal system in the gastrointestinal tract, many benzamide derivatives are effective antiemetic agents and are commonly used to control vomiting during cancer chemotherapy or radiotherapy. Costall et al., Neuropharmacoloay, 26:1321-1326 (1987). This action is almost certainly the result of an ability to block serotonin at specific sites, particularly type 3 5-hydroxytryptamine (5HT₃) receptors. Clarke et al., Trends in Pharmacological Sciences, 10:385-386 (1989). Theoretically, chemo- and radio-therapy can induce nausea and vomiting by damaging enterochromaffin cells in the gastrointestinal tract, which in turn release serotonin. Release of the neurotransmitter serotonin stimulates both afferent vagal nerve fibers (thus initiating the vomiting reflex) and serotonin receptors in the chemoreceptor trigger zone of the area postrema region of the brain. The anatomical site for this action of the benzamide derivatives, and whether such action is central (CNS), peripheral or a combination thereof, remains unresolved. Barnes et al., J. Pharm. Pharmacol., 40:586-588 (1988).

Despite the important advances in the treatment of eating disorders, particularly bulimia nervosa, that have been made during the past decade, a safe and efficacious pharmacological intervention has not been developed. Treatment of a patient often involves complex clinical decisions around such issues as choice of initial treatment modality, incomplete resolution of symptoms, and the role of long-term maintenance treatment. Thus, there remains a need for a compound effective in treating and preventing eating disorders such as bulimia.

### SUMMARY OF THE INVENTION

The present invention relates to the use of moclobemide, a metabolite of moclobemide, or a derivative of moclobemide for treating or preventing certain psychiatric and medical disorders. Moclobemide, or moclobemide metabolite, or moclobemide derivative according to this invention may be in the form of a pharmaceutically acceptable salt, hydrate, or solvate. Hereinafter, the term “moclobemide, moclobemide metabolite or moclobemide derivative,” includes moclobemide, a metabolite of moclobemide, or a derivative of moclobemide; or a prodrug of moclobemide, a metabolite of moclobemide, or a derivative of moclobemide; or pharmaceutically acceptable salt, hydrate, solvate of moclobemide, a metabolite of moclobemide, a derivative of moclobemide; or prodrug thereof. The present invention also encompasses the use of a composition in the methods of this invention, wherein the composition comprises moclobemide, a metabolite of moclobemide, or a derivative of moclobemide together with a pharmaceutically acceptable carrier (hereinafter, “moclobemide composition” or “composition”). Moclobemide metabolites according to this invention are those with MAO-A inhibitor activity, such as moclobemide-N-oxide.

The invention encompasses the use of moclobemide, a moclobemide metabolite, a moclobemide derivative or a moclobemide composition according to this invention in treating certain pain or psychogenic pain disorders, including chronic nociceptive and psychogenic pain, and related syndromes (collectively referred to herein as “pain” or “psychogenic pain disorder”). Thus, one embodiment of the present invention relates to the treatment of chronic nociceptive pain or psychogenic pain disorders by moclobemide, a moclobemide metabolite, a moclobemide derivative or a moclobemide composition.

The invention also encompasses the use of moclobemide, a moclobemide metabolite, a moclobemide derivative or a moclobemide composition in preventing chronic nociceptive or psychogenic pain disorder, such as by administration to a patient who has experienced a psychologically or physically traumatic event or who suffers from a disease commonly associated with the development of chronic nociceptive or psychogenic pain disorder. In one embodiment, the present invention contemplates the use of moclobemide, a moclobemide metabolite, a moclobemide derivative or a moclobemide composition to treat or prevent chronic nociceptive or psychogenic pain in terminally ill patients, HIV⁺ patients, AIDS patients, cancer patients, as well as patients with chronic post-surgical pain, sickle cell anemia, and rheumatoid and auto-immune disorders.

The invention encompasses the use of moclobemide, a moclobemide metabolite, a moclobemide derivative or a moclobemide composition in treating posttraumatic stress disorder (PTSD). Thus, one embodiment of the present invention relates to the treatment of PTSD by administration of a therapeutically effective amount of moclobemide, a moclobemide metabolite, a moclobemide derivative or a moclobemide composition.

The invention also encompasses the use of moclobemide, a moclobemide metabolite, a moclobemide derivative or a moclobemide composition in preventing PTSD, such as by administration to a victim of a traumatic event commonly associated with the development of PTSD, either alone or in conjunction with psychotherapy. Thus, the present invention also encompasses methods for preventing PTSD in a human by administering a therapeutically effective amount of moclobemide, a moclobemide metabolite, a moclobemide derivative or a moclobemide composition, to said human.

The invention encompasses the use of moclobemide, a moclobemide metabolite, a moclobemide derivative or a moclobemide composition in treating or preventing premenstrual dysphoric disorder (PMDD) or premenstrual syndrome (PMS). Thus, one embodiment of the present invention relates to the treatment of PMDD or PMS by administration to a female in need of treatment thereof of a therapeutically effective amount of moclobemide, a moclobemide metabolite, a moclobemide derivative or a moclobemide composition thereof.

The invention also encompasses the use of moclobemide, a moclobemide metabolite, a moclobemide derivative or a moclobemide composition to prevent PMDD or PMS. For the prevention of PMDD or PMS, moclobemide, a moclobemide metabolite, a moclobemide derivative or a moclobemide composition may be administered throughout the menstrual cycle, with administration during all weeks of the menstrual cycle except for the week following menses being preferred, and administration during the week preceding menstruation, prior to the onset of symptoms, being particularly preferred.

The invention encompasses the use of moclobemide, a moclobemide metabolite, a moclobemide derivative or a moclobemide composition in treating or preventing a sleep disorder in a mammal, particularly a human. Thus, one embodiment of the present invention relates to the treatment or prevention of sleep disorders by administration of a therapeutically effective amount of moclobemide, a moclobemide metabolite, a moclobemide derivative or a moclobemide composition.

Sleep disorders include, but are not limited to, Primary Sleep Disorders, including without limitation Primary Insomnia, Primary Hypersomnia, Narcolepsy, Circadian Rhythm Sleep Disorder, Nightmare Disorder, Sleep Terror Disorder, Sleepwalking Disorder, REM Sleep Behavior Disorder, Sleep Paralysis, and other related disorders; Substance-Induced Sleep Disorders; and Sleep Disorders Due to a General Medical Condition.

The invention encompasses the use of moclobemide, a moclobemide metabolite, a moclobemide derivative or a moclobemide composition in treating or preventing eating disorders, particularly those associated with binge eating, with bulimia being preferred. Thus, one embodiment of the present invention relates to the treatment of bulimia or another eating disorder, in particular those eating disorders associated with binge eating, by administration of a therapeutically effective amount of moclobemide, a moclobemide metabolite, a moclobemide derivative or a moclobemide composition. In another embodiment, the invention provides a method for using moclobemide, a moclobemide metabolite, a moclobemide derivative or a moclobemide composition in treating or preventing a eating disorder in a patient who is not clinically depressed as recited in DSM-IV, wherein the eating disorder to be treated does not fit the criteria of an eating disorder according to DSM-IV. Those disorders include those selected from the group consisting of carbohydrate craving, obesity or overeating.

The invention also encompasses the use of moclobemide, a moclobemide metabolite, a moclobemide derivative or a moclobemide composition in preventing bulimia or another eating disorder, such as by administration to an individual who displays symptoms or characteristics commonly associated with the development of bulimia or another eating disorder, either alone or in conjunction with psychotherapy or cognitive behavioral therapy. Thus, the present invention also encompasses methods for preventing bulimia or another eating disorder in a human by administering a therapeutically effective amount of moclobemide, a moclobemide metabolite, a moclobemide derivative or a moclobemide composition thereof, to said human.

The use of other moclobemide derivatives to treat or prevent pain and pain disorder, posttraumatic stress disorder (PTSD), premenstrual dysphoric disorder and premenstrual syndrome, certain sleep disorders, eating disorders, and the symptoms of these afflictions is also encompassed by the present invention. In particular, the use of p-iodo-N-(2-morpholinoethyl)-benzamide, p-fluoro-N-(2-morpholinoethyl)-benzamide, p-bromo-N-(2-morpholinoethyl)-benzamide, 4-chloro-N-(2-morpholinoethyl)-benzamide, and p-chloro-N-(2-morpholinoethyl)-benzamide-N′-oxide is contemplated. Each of these compounds and salts thereof is structurally similar to moclobemide and can be used with the present methods, although moclobemide is preferred.

The present invention encompasses the use of moclobemide, a moclobemide metabolite, a moclobemide derivative or a moclobemide composition to treat or prevent pain and pain disorder, posttraumatic stress disorder (PTSD), premenstrual dysphoric disorder and premenstrual syndrome, certain sleep disorders, eating disorders, and the symptoms of these afflictions in all potential human patients. In particular, the present invention contemplates the use of moclobemide, a moclobemide metabolite, a moclobemide derivative or a moclobemide composition to treat or prevent chronic nociceptive or psychogenic pain in terminally ill patients, HIV⁺ patients, AIDS patients, cancer patients, as well as patients with chronic post-surgical pain, sickle cell anemia, and rheumatoid and auto-immune disorders.

The present invention further encompasses the use of moclobemide, a moclobemide metabolite, a moclobemide derivative or a moclobemide composition in conjunction with traditional psychotherapy to treat or prevent pain and pain disorder, posttraumatic stress disorder (PTSD), premenstrual dysphoric disorder and premenstrual syndrome, certain sleep disorders, eating disorders, and the symptoms of these afflictions in a human by administering moclobemide, a moclobemide metabolite, a moclobemide derivative or a moclobemide composition to said human, before, during, or after psychotherapeutic intervention.

Furthermore, the present invention encompasses the use of moclobemide, a moclobemide metabolite, a moclobemide derivative or a moclobemide composition in combination with known anti-inflammatories or analgesics in the treatment or prevention of pain and pain disorder, posttraumatic stress disorder (PTSD), premenstrual dysphoric disorder and premenstrual syndrome, certain sleep disorders, eating disorders, and the symptoms of these afflictions.

The present invention also encompasses the use of moclobemide, a moclobemide metabolite, a moclobemide derivative or a moclobemide composition in the treatment or prevention of pain and pain disorder, posttraumatic stress disorder (PTSD), premenstrual dysphoric disorder and premenstrual syndrome, certain sleep disorders, eating disorders, and the symptoms of these afflictions in conjunction with other pharmacologically active compounds, such as known antidepressants, with tricyclic antidepressants being particularly preferred. Such known antidepressant compounds include tricyclic antidepressants such as amitriptyline, clomipramine, doxepin, imipramine, (+)-trimipramine, amoxapine, desipramine, maprotiline, nortriptyline, and protryptiline; serotonin-reuptake inhibitors such as (±)-fluoxetine, (+)-fluoxetine, fluvoxamine, paroxetine, sertraline, and (+)-venlafaxine; an optical isomer of (+)-venlafaxine; atypical antidepressants such as bupropion, nefazodone, and trazodone; and other monoamine oxidase inhibitors, such as phenelzine, tranylcypromine, and (−)-selgiline, either singly or in combination. In particular, the present invention encompasses the use of moclobemide, a moclobemide metabolite, a moclobemide derivative or a moclobemide composition in conjunction with other known antidepressants without the resulting negative drug interactions commonly associated with MAOI's.

### DETAILED DESCRIPTION OF THE INVENTION

The present invention encompasses a method of treating pain in a human, which comprises administering to said human suffering from pain a therapeutically effective amount of moclobemide, a moclobemide metabolite, a moclobemide derivative or a moclobemide composition.

The present invention also encompasses a method of preventing pain in a human who has experienced a physically traumatic event or who suffers from a disease commonly associated with the development of pain, which comprises administering to said human a therapeutically effective amount of moclobemide, a moclobemide metabolite, a moclobemide derivative or a moclobemide composition. The present invention also encompasses a method of treating psychogenic pain disorder in a human, which comprises administering to said human suffering from pain a therapeutically effective amount of moclobemide, a moclobemide metabolite, a moclobemide derivative or a moclobemide composition. In one embodiment, the chronic nociceptive pain is treated in a patient having cancer, AIDS, chronic post surgical pain, sickle cell anemia, or an auto-immune disorder. In another embodiment of this invention the patient having chronic nocioceptive pain does not have nerve tissue damage.

The present invention also encompasses a method of preventing psychogenic pain disorder in a human who has experienced a psychologically or physically traumatic event or who suffers from a disease commonly associated with the development of psychogenic pain disorder, which comprises administering to said human a therapeutically effective amount of moclobemide, a moclobemide metabolite, a moclobemide derivative or a moclobemide composition.

The present invention encompasses a method of treating PTSD in a human, which comprises administering to said human suffering from PTSD a therapeutically effective amount of moclobemide, a moclobemide metabolite, a moclobemide derivative or a moclobemide composition.

The present invention further encompasses a method of treating PTSD in a human, which comprises administering to said human suffering from PTSD a therapeutically effective amount of moclobemide, a moclobemide metabolite, a moclobemide derivative or a moclobemide composition, either alone or in conjunction with psychotherapy, in a manner designed to prevent, minimize, or eliminate any negative psychological or physiological connection between the stimuli that precipitate an episode of PTSD and the traumatic event.

The present invention also encompasses a method of preventing PTSD in a human who is a victim of a traumatic event commonly associated with the development of PTSD, which comprises administering to said human suffering from PTSD a therapeutically effective amount of moclobemide, a moclobemide metabolite, a moclobemide derivative or a moclobemide composition.

The present invention further encompasses a method of preventing PTSD in a human who is a victim of a traumatic event commonly associated with the development of PTSD, which comprises administering to said human suffering from PTSD a therapeutically effective amount of moclobemide, a moclobemide metabolite, a moclobemide derivative or a moclobemide composition, either alone or in conjunction with psychotherapy, in a manner designed to prevent, minimize, or eliminate any negative psychological or physiological connection between the trigger and the traumatic event and prevent the onset of PTSD, said amount being sufficient to prevent said PTSD.

The present invention encompasses a method of treating PMDD or PMS in a female, which comprises administering to said female a therapeutically effective amount of moclobemide, a moclobemide metabolite, a moclobemide derivative or a moclobemide composition.

The present invention also encompasses a method of preventing PMDD or PMS in a female, which comprises administering to said female a therapeutically effective amount of moclobemide, a moclobemide metabolite, a moclobemide derivative or a moclobemide composition prior to and/or during the last week of the luteal phase of the menstrual cycle, in a manner designed to prevent, minimize, or eliminate the onset of PMDD or PMS symptoms.

The present invention encompasses a method of treating a sleep disorder in a human, which comprises administering to said human suffering from a sleep disorder a therapeutically effective amount of moclobemide, a moclobemide metabolite, a moclobemide derivative or a moclobemide composition thereof.

The present invention also encompasses a method of preventing a sleep disorder in a human who displays symptoms commonly associated with the development of a sleep disorder, or who is environmentally or genetically predisposed to or at risk for a sleep disorder, which comprises administering to said human a therapeutically effective amount of moclobemide, a moclobemide metabolite, a moclobemide derivative or a moclobemide composition.

The present invention further encompasses a method of treating or preventing a sleep disorder in a human, which comprises administering to said human suffering from a sleep disorder or in need of prevention thereof a therapeutically effective amount of moclobemide, a moclobemide metabolite, a moclobemide derivative or a moclobemide composition thereof, in conjunction with psychotherapy, in a manner designed to prevent, minimize, or eliminate any psychological factors contributing to the development or persistence of the sleep disorder.

As used herein, the term “sleep disorder” includes Primary Sleep Disorders, including without limitation Primary Insomnia, Primary Hypersomnia, Narcolepsy, Circadian Rhythm Sleep Disorder, Nightmare Disorder, Sleep Terror Disorder, Sleepwalking Disorder, REM Sleep Behavior Disorder, Sleep Paralysis, and other related disorders; Substance-Induced Sleep Disorders; and Sleep Disorders Due to a General Medical Condition.

In a preferred embodiment, the invention encompasses the treatment or prevention of narcolepsy in a human which comprises administering to said human a therapeutically effective amount of moclobemide, a moclobemide metabolite, a moclobemide derivative or a moclobemide composition.

The present invention encompasses a method of treating bulimia or another eating disorder in a human, which comprises administering to said human suffering from said eating disorder a therapeutically effective amount of moclobemide, a moclobemide metabolite, a moclobemide derivative or a moclobemide composition.

The present invention further encompasses a method of treating bulimia or another eating disorder in a human, which comprises administering to said human suffering from bulimia or another eating disorder a therapeutically effective amount of moclobemide, a moclobemide metabolite, a moclobemide derivative or a moclobemide composition, either alone or in conjunction with psychotherapy or cognitive behavioral therapy, in a manner designed to prevent, minimize, or eliminate any psychological aspects of said eating disorder.

The present invention also encompasses a method of preventing bulimia or another eating disorder in a human who exhibits characteristics or symptoms commonly associated with the development of an eating disorder, which comprises administering to said human a therapeutically effective amount of moclobemide, a moclobemide metabolite, a moclobemide derivative or a moclobemide composition.

The present invention further encompasses a method of preventing bulimia or another eating disorder in a human who exhibits characteristics or symptoms commonly associated with the development of an eating disorder such as bulimia, which comprises administering to said human a therapeutically effective amount of moclobemide, a moclobemide metabolite, a moclobemide derivative or a moclobemide composition, in conjunction with psychotherapy or cognitive behavioral therapy, in a manner designed to prevent, minimize, or eliminate any psychological aspects of said eating disorder and prevent the onset of said eating disorder, said amount being sufficient to prevent said eating disorder. In one embodiment, the human being treated for the eating disorder is not also clinically depressed according to DSM-IV.

Moclobemide, as well as certain other moclobemide derivatives, can be synthesized according to the method described in U.S. Pat. No. 4,210,754 to Burkard et al., and in U.S. Pat. No. 4,906,626 to Amrein et al., which are incorporated by reference herein in their entirety.

The moclobemide metabolite known as moclobemide-N-oxide, which can be represented by the formula:

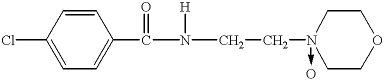

has been shown to have MAO-A inhibitory activity, and its use in the treatment or prevention of an affliction embodied in the group consisting of pain and pain disorder, posttraumatic stress disorder (PTSD), premenstrual dysphoric disorder and premenstrual syndrome, certain sleep disorders, eating disorders, and the symptoms of these afflictions in a human is also encompassed by the present invention.

Prodrugs, i.e. drugs that are metabolized in vivo into the active agent, and methods for making prodrugs are readily know in the art (e.g., Balant, L. P., “Prodrugs for the Improvement of Drug Absorption Via Different Routes of Administration,” Eur. J. Drug Metab. Pharmacokinet. 15:143-153 (1990); and Bundgaard, H., “Novel Chemical Approaches in Prodrug Design,” Drugs of the Future 16:443-458 (1991); incorporated by reference herein). In one embodiment, derivatives according to this invention have MAOI activity.

The magnitude of a prophylactic or therapeutic dose of the active ingredient (e.g., moclobemide, a moclobemide metabolite, a moclobemide derivative) in the prevention or the acute or chronic management of an affliction embodied in the group consisting of pain and pain disorder, posttraumatic stress disorder (PTSD), premenstrual dysphoric disorder and premenstrual syndrome, certain sleep disorders, eating disorders, and the symptoms of these afflictions in a human will vary with the severity of the patient's affliction and the route of administration. The dose and dose frequency will also vary according to the age, weight and response of the individual patient. In general, the recommended daily dose range for the conditions described herein lies within the range of from about 50 mg to about 1200 mg per day, generally divided equally into doses given one to four times a day. Preferably, a daily dose range should be between 150 mg and 900 mg per day, usually divided equally into a two to four times a day dosing. Most preferably, a daily dose range should be between 150 mg and 600 mg per day, usually divided equally into a two to four times a day dosing. It may be necessary to use dosages outside these ranges in some cases, and the treating physician will know how to increase, decrease or interrupt treatment based upon patient response. The various terms described above such as “therapeutically effective amount,” are encompassed by the above-described dosage amounts and dose frequency schedule.

For use in treating or preventing an affliction embodied in the group consisting of pain and pain disorder, posttraumatic stress disorder (PTSD), premenstrual dysphoric disorder and premenstrual syndrome, certain sleep disorders, eating disorders, and the symptoms of these afflictions in a human, the physician will generally prescribe the period of treatment and frequency of dose of moclobemide, a moclobemide metabolite, a moclobemide derivative or a moclobemide composition on a patient-by-patient basis. In general, however, treatment or prevention of an affliction embodied in the group consisting of pain and pain disorder, posttraumatic stress disorder (PTSD), premenstrual dysphoric disorder and premenstrual syndrome, certain sleep disorders, eating disorders, and the symptoms of these afflictions in a human with moclobemide, a moclobemide metabolite, a moclobemide derivative or a moclobemide composition may be carried out for as long a period as necessary, either in a single, uninterrupted session, or in discrete sessions timed to coincide with exposure to biochemical, environmental, or hormonal stimuli likely to trigger symptoms.

For example, treatment or prevention of chronic nociceptive or psychogenic pain disorder may be timed to coincide with episodes of clinically significant pain. For example, treatment or prevention of PTSD may be timed to coincide with exposure to stimuli associated with the traumatic event likely to trigger symptoms of anxiety or increased arousal. For example, treatment for sleep disorders may be timed to coincide with acute episodes of sleep disorder, or with exposure to stimuli associated with the onset of an episode of sleep disorder.

Most preferably, moclobemide, a moclobemide metabolite, a moclobemide derivative or a moclobemide composition therapy may be carried out for a period of at least 4 weeks.

Moclobemide, a moclobemide metabolite, a moclobemide derivative or a moclobemide composition may also be administered before, along with, or after traditional psychotherapy, in particular cognitive behavioral therapy. Thus, moclobemide, a moclobemide metabolite, a moclobemide derivative or a moclobemide composition may be utilized in accordance with the present invention as an adjunct to conventional behavioral therapy that aims to eliminate, minimize, or prevent symptoms commonly associated with an affliction embodied in the group consisting of pain and pain disorder, posttraumatic stress disorder (PTSD), premenstrual dysphoric disorder and premenstrual syndrome, certain sleep disorders, eating disorders, and the symptoms of these afflictions in a human.

For example, moclobemide, a moclobemide metabolite, a moclobemide derivative or a moclobemide composition may be utilized in accordance with the present invention as an adjunct to conventional behavioral therapy that aims to eliminate, minimize, or prevent the formation of negative associations between a traumatic event commonly associated with the development of PTSD and stimuli that represent or symbolize that event. For example, survivors of wartime imprisonment in cold climates have been known to experience episodes of PTSD upon subsequent exposure to similar climates. DSM-IV, p. 424. Behavioral therapies well known to those of skill in the art typically attempt to eliminate the psychological and physiological symptoms associated with PTSD and precipitated by exposure to the triggering stimulus, the climate in this example, by forcing the patient to confront the stimulus in a progressively more intense manner. Repeated interaction with the stimulus is thought to decrease the threat it represents to the patient, and eliminate or minimize the “Pavlovian” response thereto. The concomitant administration of moclobemide, a moclobemide metabolite, a moclobemide derivative or a moclobemide composition can be used to facilitate this process, in either a treatment or prevention setting, by pharmacologically reducing the negative sensations experienced by the patient, thus reducing the negative psychological connotations of the stimulus. When used for treatment, the stimuli would be chosen based on the patient's expressed anxieties. When used for prevention, the stimuli would be chosen by the treating psychologist or physician from among those most commonly associated with the development of PTSD in victims of similar traumatic events.

Any suitable route of administration may be employed for providing the patient with an effective dosage of moclobemide, a moclobemide metabolite, a moclobemide derivative or a moclobemide composition. For example, oral, rectal, parenteral, transdermal, subcutaneous, sublingual, intranasal, intramuscular, intrathecal and the like may be employed as appropriate. Dosage forms include tablets, coated tablets, caplets, capsules (e.g., hard gelatin capsules), troches, dragees, dispersions, suspensions, solutions, patches and the like, including sustained release formulations well known in the art. See, e.g., Introduction to Pharmaceutical Dosage Forms, 1985, Ansel, H. C., Lea and Febiger, Philadelphia, Pa.; Remington's Pharmaceutical Sciences, 1995, Mack Publ. Co., Easton, Pa.

The compositions of the present invention may also comprise a pharmaceutically acceptable carrier and optionally other therapeutic ingredients. The term “pharmaceutically acceptable salt” refers to salts prepared from pharmaceutically acceptable non-toxic acids including inorganic acids and organic acids.

Since the compound of the present invention is basic, salts may be prepared from pharmaceutically acceptable non-toxic acids including inorganic and organic acids. Such acids include maleic, acetic, benzene-sulfonic (besylate), benzoic, camphorsulfonic, citric, ethenesulfonic, fumaric, gluconic, glutamic, hydrobromic, hydrochloric, isethionic, lactic, malic, mandelic, methanesulfonic, mucic, nitric, pamoic, pantothenic, phosphoric, succinic, sulfuric, tartaric, p-toluenesulfonic acid and the like. Particularly preferred are hydrobromic, hydrochloric, maleic, phosphoric, and sulfuric acids.

The compositions include compositions suitable for oral, rectal, transdermal, sublingual, and parenteral administration (including subcutaneous, intramuscular, intrathecal and intravenous), although the most suitable route in any given case will depend on the nature and severity of the condition being treated. The most preferred route of administration of the present invention is the oral route. The composition may be conveniently presented in unit dosage form and prepared by any of the methods well-known in the art of pharmacy.

In the case where an oral composition is employed, a suitable dosage range for use is, e.g., from about 50 mg to about 1200 mg per day, generally divided equally into a one to four times a day dosing, preferably from about 150 mg to about 900 mg per day, generally divided equally into a two to four times a day dosing and most preferably from about 150 mg to about 600 mg per day, generally divided equally into a two to four times a day dosing. Patients may be upward titrated from below to within this dose range to achieve satisfactory control or prevention of symptoms as appropriate.

In practical use, moclobemide, a moclobemide metabolite, or a moclobemide derivative can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous injections or infusions). In preparing the compositions for oral dosage form, any of the usual pharmaceutical media may be employed, for example, water, glycols, oils, alcohols, flavoring agents, preservatives, coloring agents and the like in the case of oral liquid preparations, for example, suspensions, elixirs and solutions; or aerosols; or carriers such as starches, sugars, microcrystalline cellulose, stabilizers, diluents, granulating agents, lubricants, binders, fillers, disintegrating agents and the like in the case of oral solid preparations such as, powders, capsules and tablets, with the solid oral preparations being preferred over the liquid preparations. The preferred solid oral preparation is tablets. The most preferred solid oral preparation is coated tablets. Because of their ease of administration tablets and capsules represent the most advantageous oral dosage unit form, in which case solid pharmaceutical carriers are obviously employed. If desired, tablets may be coated by standard aqueous or nonaqueous techniques. The preparation of coated tablets, sachets, and hard gelatin capsules containing moclobemide as the active ingredient is described in U.S. Pat. No. 4,906,626, which is incorporated herein by reference.

In addition to the common dosage forms set out above, the compounds of the present invention may also be administered by controlled release or sustained release means and/or delivery devices such as those described in U.S. Pat. Nos. 3,845,770; 3,916,899; 3,536,809; 3,598,123; 3,630,200, 4,008,719, 4,687,660, and 4,769,027, the disclosures of which are hereby incorporated by reference. Preferred controlled release or sustained release tablets suitable for use with moclobemide are described in U.S. Pat. No. 5,427,798, which is incorporated herein by reference.

Pharmaceutical stabilizers may also be used to stabilize compositions comprising moclobemide, a moclobemide metabolite, or a moclobemide derivative; acceptable stabilizers include but are not limited to L-cysteine hydrochloride, glycine hydrochloride, malic acid, sodium metabisulfite, citric acid, tartaric acid and L-cysteine dihydrochloride. See, e.g., U.S. Pat. No. 5,358,970, which is incorporated herein by reference.

Compositions of the present invention suitable for oral administration may be presented as discrete units such as capsules, cachets, or tablets or aerosol sprays, each containing a predetermined amount of the active ingredient, as a powder or granules or as a solution or a suspension in an aqueous liquid, a non-aqueous liquid, an oil-in-water emulsion, or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine the active ingredient in a free-flowing form such as powder or granules, optionally mixed with one or more of a binder, filler, stabilizer, lubricant, inert diluent, and/or surface active or dispersing agent. Molded tablets may be made by molding in a suitable machine a mixture of the powdered compound moistened with an inert liquid diluent. Desirably, each tablet contains from about 50 mg to about 300 mg of the active ingredient, and each cachet or capsule contains from about 50 mg to about 300 mg of the active ingredient. In a preferred embodiment, the tablet, cachet or capsule contains one of four dosages: about 50 mg, about 75 mg, about 100 mg, and about 150 mg of active ingredient.

Throughout this specification and claims, the word “comprise,” or variations such as “comprises” or “comprising,” will be understood to imply the inclusion of a stated integer or group of integers but not the exclusion of any other integer or group of integers.

The invention is further defined by reference to the following examples describing in detail the preparation of the compound and compositions of the present invention. It will be apparent to those skilled in the art that many modifications, both to materials and to methods, may be practiced without departing from the purpose and interest of this invention.

### EXAMPLES

### Example 1

ORAL FORMULATION
Coated Tablets:

| Formula | Quantity per Tablet (mg.) |
|---|---|
| moclobemide | 50.0 |
| Lactose | 74.0 |
| Corn Starch | 35.0 |
| Water (per thousand | 30.0 ml* |
| Tablets) | |
| Magnesium Stearate | 1.0 |
| Corn Starch | 25.0 |

*The water evaporates during manufacture.

The active ingredient (moclobemide) is blended with the lactose until a uniform blend is formed. The smaller quantity of corn starch is blended with a suitable quantity of water to form a corn starch paste. This is then mixed with said uniform blend until a uniform wet mass is formed. The remaining corn starch is added to the resulting wet mass and mixed until uniform granules are obtained. The granules are then screened through a suitable milling machine, using a ¼ inch stainless steel screen. The milled granules are then dried in a suitable drying oven until the desired moisture content is obtained. The dried granules are then milled through a suitable milling machine using ¼ mesh stainless steel screen. The magnesium stearate is then blended and the resulting mixture is compressed into tablets of desired shape, thickness, hardness and disintegration.

Tablets are coated by standard aqueous or nonaqueous techniques. For example, 2.5 mg of hydroxypropymethylcellulose can be dissolved in 25 mg of deionized water. An aqueous (10 mg) suspension of 1.88 mg talc, 0.5 mg of titanium dioxide, 0.1 mg of yellow iron oxide, and 0.02 mg of red iron oxide is stirred into this solution.

The coating suspension is sprayed on the tablets and the coated tablets are dried overnight at 45° C.

### Example 2

ORAL FORMULATION
Capsules:

Quantity per capsule in mg.

| Formula | A | B | C |
|---|---|---|---|
| Active ingredient | 25 | 50 | 75 |
| moclobemide | | | |
| Lactose | 149.5 | 124.5 | 374.0 |
| Corn Starch | 25.0 | 25.0 | 50.0 |
| Magnesium Stearate | 0.5 | 0.5 | 1.0 |
| Compression Weight | 200.0 | 200.0 | 500.0 |

The active ingredient (moclobemide), lactose, and corn starch are blended until uniform; then the magnesium stearate is blended into the resulting powder. The resulting mixture is encapsulated into suitably sized two-piece hard gelatin capsules.

### Example 3

ORAL FORMULATION
Tablets

Quantity per Tablet in mg.

| Formula | A | B | C |
|---|---|---|---|
| Active ingredient, | 20 | 40 | 100 |
| moclobemide | | | |
| lactose BP | 134.5 | 114.5 | 309.0 |
| starch BP | 30.0 | 30.0 | 60.0 |
| Pre-gelatinized Maize | 15.0 | 15.0 | 30.0 |
| Starch BP | | | |
| magnesium stearate | 0.5 | 0.5 | 1.0 |
| Compression Weight | 200.0 | 200.0 | 500.0 |

The active ingredient (moclobemide) is sieved through a suitable sieve and blended with lactose, starch, and pre-gelatinized maize starch. Suitable volumes of purified water are added and the powders are granulated. After drying, the granules are screened and blended with the magnesium stearate. The granules are then compressed into tablets using punches.

Tablets of other strengths may be prepared by altering the ratio of active ingredient to lactose or the compression weight and using punches to suit. In particular, single unit dosage forms of moclobemide in 50, 100, 150, and 200 mg are preferred and can be easily manufactured by those of skill in the art. For example, tablets of the following composition, as described in U.S. Pat. No. 4,210,754, incorporated herein in its entirety, may be prepared by methods known to those of skill in the art:

Tablets

| Formula | Quantity per Tablet in mg. |
|---|---|
| Active ingredient, | 50.0 |
| moclobemide | |
| Lactose | 95.0 |
| Maize starch | 100.0 |
| Talc | 4.5 |
| Magnesium stearate | 0.5 |
| Weight per tablet | 250.0 |

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims.

The contents of all references described herein are hereby incorporated by reference. Other embodiments are within the following claims.

What is claimed is:

1. A method for treating chronic nociceptive pain in a human, which comprises administering to a human in need of treatment for chronic nociceptive pain a therapeutically effective amount of moclobemide, a moclobemide metabolite, a moclobemide derivative or a moclobemide composition.

2. A method for preventing chronic nociceptive pain in a human who has experienced a physically or psychologically traumatic event or who suffers from a disease commonly associated with the development of pain which comprises administering to said human in need of prevention of pain a therapeutically effective amount of moclobemide, a moclobemide metabolite, a moclobemide derivative or a moclobemide composition, said amount being sufficient to prevent said pain.

3. A method for treating psychogenic pain disorder in a human, which comprises administering to a human in need of treatment for pain disorder a therapeutically effective amount of moclobemide, a moclobemide metabolite, a moclobemide derivative or a moclobemide composition.

4. A method for preventing psychogenic pain disorder in a human who has experienced a physically or psychologically traumatic event or who suffers from a disease commonly associated with the development of pain disorder which comprises administering to said human in need of prevention of pain a therapeutically effective amount of moclobemide, a moclobemide metabolite, a moclobemide derivative or a moclobemide composition, said amount being sufficient to prevent said pain.

5. A method for treating posttraumatic stress disorder in a human, which comprises administering to a human in need of treatment for posttraumatic stress disorder a therapeutically effective amount of moclobemide, a moclobemide metabolite, a moclobemide derivative or a moclobemide composition.

6. A method for preventing posttraumatic stress disorder in a human who has been exposed to a traumatic event commonly associated with the development of posttraumatic stress disorder which comprises administering to said human in need of prevention of posttraumatic stress disorder a therapeutically effective amount of moclobemide, a moclobemide metabolite, a moclobemide derivative or a moclobemide composition, said amount being sufficient to prevent said posttraumatic stress disorder.

7. A method for treating premenstrual dysphoric disorder or premenstrual syndrome in a female in need of treatment thereof, which comprises administering to a female in need of treatment for premenstrual dysphoric disorder or premenstrual syndrome a therapeutically effective amount of moclobemide, a moclobemide metabolite, a moclobemide derivative or a moclobemide composition.

8. A method for preventing premenstrual dysphoric disorder or premenstrual syndrome in a female, which comprises administering to a female in need of prevention of premenstrual dysphoric disorder or premenstrual syndrome a therapeutically effective amount of moclobemide, a moclobemide metabolite, a moclobemide derivative or a moclobemide composition.

9. A method for treating or preventing a sleep disorder in a human, which comprises administering to a human in need of prevention of or treatment for a sleep disorder a therapeutically effective amount of moclobemide, a moclobemide metabolite, a moclobemide derivative or a moclobemide composition.

10. A method for treating an eating disorder, which comprises administering to a human in need of treatment for an eating disorder a therapeutically effective amount of moclobemide, a moclobemide metabolite, a moclobemide derivative or a moclobemide composition.

11. A method for preventing an eating disorder, which comprises administering to a human in need of prevention of an eating disorder a therapeutically effective amount of moclobemide, a moclobemide metabolite, a moclobemide derivative or a moclobemide composition.

12. The method of claim 5 or 6 further comprising treating said human with psychotherapy designed to eliminate, minimize, or prevent negative psychological or physiological associations of stimuli representing or symbolic of the traumatic event.

13. The method according to claim 7 or 8 wherein said administration continues throughout the menstrual cycle but ceases during the week following menses.

14. The method according to claim 7 wherein said administration begins during the last week of the luteal phase and continues until after the onset of menses.

15. The method according to claim 8 wherein said administration begins prior to the last week of the luteal phase and terminates with the onset of menses.

16. The method of claim 9 wherein the sleep disorder is a primary sleep disorder.

17. The method of claim 16 wherein the primary sleep disorder is selected from the group consisting of primary insomnia, primary hypersomnia, narcolepsy, circadian rhythm sleep disorder, nightmare disorder, sleep terror, and sleepwalking disorder.

18. The method of claim 17 wherein the primary sleep disorder is narcolepsy.

19. The method of claim 9 wherein the sleep disorder is substance-induced sleep disorder.

20. The method of claim 9 wherein the sleep disorder is sleep disorder due to a general medical condition.

21. The method according to claim 10 or claim 11 wherein the eating disorder is bulimia nervosa.

22. The method according to claim 10 or claim 11 wherein the eating disorder is binge eating disorder.

23. The method according to claim 10 or claim 11 wherein the eating disorder is anorexia nervosa.

24. The method according to claim 10 or claim 11, wherein said human is not clinically depressed.

25. The method according to claim 1 or claim 2 wherein the human has cancer.

26. The method according to claim 1 or claim 2 wherein the human has human immunodeficiency virus.

27. The method according to claim 1 or claim 2 wherein the human has acquired immunodeficiency syndrome.

28. The method according to claim 1 or claim 2 wherein the human is terminally ill.

29. The method according to claim 1 or claim 2 wherein the human has chronic post-surgical pain.

30. The method according to claim 1 or claim 2 wherein the human has sickle cell anemia.

31. The method according to claim 1 or claim 2 wherein the human has an auto-immune disorder.

32. The method according to any one of claims 1-11 wherein moclobemide, a moclobemide metabolite, a moclobemide derivative or a moclobemide composition is administered orally or parentally.

33. The method according to any one of claims 1-11 wherein moclobemide, a moclobemide metabolite, a moclobemide derivative or a moclobemide composition is administered orally as a tablet or a capsule.

34. The method according to any one of claims 1-11 wherein moclobemide, a moclobemide metabolite, a moclobemide derivative or a moclobemide composition is administered transdermally as a transdermal patch.

35. The method according to any one of claims 1-11 wherein the amount administered is from about 50 mg to about 1200 mg.

36. The method according to any one of claims 1-11 wherein the amount administered is from about 150 mg to about 900 mg.

37. The method according to claim 36 wherein the amount administered is from about 150 mg to about 600 mg.

38. The method according to any one of claims 1-11 wherein the amount of moclobemide, a moclobemide metabolite, a moclobemide derivative or a moclobemide composition is administered together with a pharmaceutically acceptable carrier.

39. The method according to any one of claims 1-11 wherein moclobemide is administered as the hydrochloride salt.

40. The method according to any one of claims 1-11 wherein moclobemide, a moclobemide metabolite, a moclobemide derivative or a moclobemide composition is administered in a sustained or controlled release formulation.

41. The method according to any one of claims 1-11 wherein said administration is made one to four times per day.

42. The method according to any one of claims 1-11 wherein said administration continues for a period of at least 4 weeks.

43. The method according to any one of claims 1-11 further comprising treating said human with psychotherapy designed to eliminate, minimize, or prevent negative psychological or physiological associations.

44. The method according to any one of claims 1-11 further comprising treating said human with an antidepressant.

45. The method according to claim 44 wherein the antidepressant is a tricyclic antidepressant.

46. The method according to claim 45 wherein the tricyclic antidepressant is selected from the group consisting of amitriptyline, clomipramine, doxepin, imipramine,(+)-trimipramine, amoxapine, desipramine, maprotiline, nortriptyline, and protryptiline.

47. The method according to claim 44 wherein the antidepressant is selected from the group consisting of (±)-fluoxetine, (+)-fluoxetine, fluvoxamine, paroxetine, sertraline, (±)-venlafaxine, an active optical isomer of (+)-venlafaxine, bupropion, nefazodone, trazodone, phenelzine, tranylcypromine, and (−)-selegiline.

48. The method according to any one of claims 1-11, wherein the moclobemide derivative is selected from the group consisting of p-iodo-N-(2-morpholinoethyl)-benzamide, p-fluoro-N-(2-morpholinoethyl)-benzamide, p-bromo-N-(2-morpholinoethyl)-benzamide, p-chloro-N-(2-morpholinoethyl)-benzamide, a benzamide derivative thereof, or p-chloro-N-(2-morpholinoethyl)-benzamide-N′-oxide.

49. The method according to any one of claims 1-4, wherein psychological factors play a major role in the onset, severity, exacerbation, or maintenance of the pain.
